(19) United States
(12) Patent Application Publication
(10) Pub. No.: US 2026/0158191 A1
(43) Pub. Date: Jun. 11, 2026

(54) METHOD FOR CREATING FORMABLE CARTILAGE TISSUE WITHOUT REQUIRING SCAFFOLD

(71) Applicant: Public University Corporation Yokohama City University, Yokohama-shi, Kanagawa (JP)

(72) Inventors: Hideki TANIGUCHI, Yokohamashi, Kanagawa (JP); Soichiro MURATA, Yokohamashi, Kanagawa (JP); Takayoshi OBA, Yokohamashi, Kanagawa (JP)

(73) Assignee: Public University Corporation Yokohama City University, Yokohama-shi, Kanagawa (JP)

(21) Appl. No.: 18/685,860

(22) PCT Filed: Jun. 27, 2022
(86) PCT No.: PCT/JP2022/025582

§ 371 Date: Feb. 22, 2024

(30) Foreign Application Priority Data
Aug. 31, 2021 (JP) 2021-141210

(51) Int. Cl. A61L 27/38; C12N 5/077

(52) U.S. Cl. CPC A61L 27/3817; C12N 5/0655; C12N 2501/115; C12N 2501/15; C12N 2501/155; C12N 2501/415; C12N 2506/02; C12N 2506/45; C12N 2539/00

(57) ABSTRACT

The present invention develops a method for creating a cartilage tissue that solves the following problems.
1. Chondrocyte differentiation is not hindered by shape making.
2. The cartilage tissue is capable of being made into a desired shape without using scaffolds.

Provided is a method for creating an artificial cartilage tissue, comprising making chondrocyte progenitor cell-containing spheroids into a desired shape while seeding the spheroids on a support; culturing the spheroids while feeding a culture medium from both the obverse and reverse sides of the spheroid-seeded surface to thereby perform fusion among the spheroids; and maturing the resultant fused spheroids into a cartilage tissue in vitro.

### TECHNICAL FIELD

The present invention relates to a method for creating a shapeable cartilage tissue not requiring a scaffold.

### BACKGROUND ART

Conventionally, fundamental treatment for dyschondroplasia in the craniofacial area and osteochondritis dissecans and traumatic cartilage damage in limbs has been autologous cartilage transplantation. This method has such drawbacks that collectable cartilage is limited and that postoperative pain occurs at the site of collection. Hence, a method for cultured cartilage transplantation has been developed in which a small quantity of stem cells is cultured as they are expanded through directed differentiation and then transplanted.

After the development of a cartilage combined with an artificial scaffold material such as hydrogel, a more physiological, scaffold-free cartilage not requiring scaffold materials was developed (Non-Patent Document No. 1). However, since this scaffold-free cartilage is obtained by aggregating cell masses and subjecting the resultant cell masses to three-dimensional suspension-culture, it has been impossible to make the cartilage into a desired shape. Since it is important that a cartilage tissue to be transplanted into a patient conforms to a required shape, non-shapeability is one of those problems that should be overcome most urgently.

### PRIOR ART LITERATURE

### Non-Patent Documents

Non-Patent Document No. 1: Enomura M, et al., Int J Mol Sci. 2020, 21, 8496

### DISCLOSURE OF THE INVENTION

### Problem for Solution by the Invention

To date, a scaffold-free and yet shapeable cartilage has never been developed. The present invention aims at developing a method for creating a cartilage tissue which would overcome the following problems:
1. Chondrocyte differentiation is not hindered by shape making.
2. The cartilage tissue is capable of being made into a desired shape without using scaffolds.

### Means to Solve the Problem

For creating a shapeable and yet scaffold-free cartilage tissue from chondrocyte progenitor cells, the present inventors considered that the following method would be desirable: chondrocyte progenitor cells are first aggregated to form small-sized spheroids which are then re-assembled to form an intended shape. However, if chondrocyte progenitor cells are aggregated to form spheroids, cell death due to undernutrition occurs in spheroids 300 μm or more in diameter because they are not supplied with sufficient oxygen. Further, those spheroids adhering to the bottom of plates may have a possibility that the adhered surface will undergo undernutrition or hypoxia. In order to solve these problems, the present inventors seeded and aggregated chondrocyte progenitor cells on micro-pattern plates to thereby prepare groups of spheroids 200 μm in diameter. The thus prepared spheroids were arranged in a desired shape and fused to each other. Since a fused spheroid is larger than a single cell by about 100 times and identifiable even with eyes, those spheroids are capable of forming a three-dimensional structure in such a manner that balls are piled up. To this end, operations were performed on cell culture inserts and the lower parts thereof were filled with a culture medium so that spheroids in contact with the bottom would not undergo undernutrition or hypoxia. A cartilage tissue prepared by this method will be capable of omnidirectional medium or gas exchange. A cartilage tissue prepared by culturing for 15-30 days according to this method became a mature cartilage when transplanted into a living body. Further, a cartilage tissue cultured for 56-70 days according to this method became a hypertrophic cartilage, which then became a bone tissue when transplanted into a living body. The present invention has been achieved based on these findings.

The gist of the present invention is as follows.
(1) A method for creating an artificial cartilage tissue, comprising making chondrocyte progenitor cell-containing spheroids into a desired shape while seeding the spheroids on a support; culturing the spheroids while feeding a culture medium from both the obverse and reverse sides of the spheroid-seeded surface to thereby perform fusion among the spheroids; and maturing the resultant fused spheroids into a cartilage tissue in vitro.
(2) The method of (1) above, wherein the chondrocyte progenitor cell is a cell induced for differentiation from embryonic stem cells and/or induced pluripotent stem cells.
(3) The method of (1) above, wherein the chondrocyte progenitor cell is a cell induced for differentiation from perichondrocytes collected from the perichondrium.
(4) The method of any one of (1) to (3) above, wherein the chondrocyte progenitor cell-containing spheroid is 20-1000 μm in diameter.
(5) The method of any one of (1) to (4) above, wherein one spheroid contains 100-7500 chondrocyte progenitor cells.
(6) The method of any one of (1) to (5) above, wherein the chondrocyte progenitor cell-containing spheroid has been prepared by culturing chondrocyte progenitor cells in a culture vessel having a non-cell-adhesive surface.
(7) The method of any one of (1) to (6) above, wherein the chondrocyte progenitor cell-containing spheroid has been prepared by culturing chondrocyte progenitor cells in a culture medium containing TGF-β, bFGF and Wnt/β-catenin inhibitor.
(8) The method of any one of (1) to (7) above, wherein the fused spheroid is matured into a cartilage tissue by culturing in a culture medium containing BMP.
(9) The method of any one of (1) to (8) above, wherein the time period of culture of the fused spheroid for maturing into a cartilage tissue is 14-42 days.
(10) The method of any one of (1) to (8) above, wherein the time period of culture of the fused spheroid for maturing into a cartilage tissue is 42-84 days.
(11) The method of any one of (1) to (10) above, further comprising transplanting the cartilage tissue matured in vitro into a non-human animal and maturing the cartilage tissue furthermore.
(12) An artificial cartilage tissue prepared by the method of any one of (1) to (11) above, which is 6 mm or more in diameter and 0.5 mm or more in thickness.
(13) An artificial cartilage tissue prepared by the method of (10) above, wherein a part or the whole of the cartilage tissue is differentiated into bone tissue after transplantation into a living body.
(14) A composition comprising an artificial cartilage tissue prepared by the method of any one of (1) to (11) above, which is used for transplantation into a living body to compensate for a deficiency of cartilage tissue and/or bone tissue therein.
(15) The composition of (14) above comprising an artificial cartilage tissue prepared by the method of (9) above, which is used for transplantation into a living body to compensate for a deficiency of cartilage tissue therein.
(16) The composition of (14) above comprising an artificial cartilage tissue prepared by the method of (10) above, which is used for transplantation into a living body to compensate for a deficiency of bone tissue therein.
(17) A method for preparing an artificial bone tissue, comprising transplanting an artificial cartilage tissue prepared by the method of (10) above into a non-human animal and maturing the cartilage tissue into a bone tissue.
(18) An artificial bone tissue prepared by the method of (17) above, which is 6 mm or more in diameter and 0.5 mm or more in thickness.
(19) A composition comprising an artificial bone tissue prepared by the method of (17) above, which is used for transplantation into a living body to compensate for a deficiency of bone tissue therein.

### Effect of the Invention

According to the present invention, it becomes possible to prepare in vitro a cartilage tissue of any shape having a strength sufficient for transplantation. By transplanting an artificial cartilage tissue prepared by the method of the present invention into a living body, a matured cartilage or bone tissue can be obtained.

The present specification encompasses the contents disclosed in the specification and/or drawings of Japanese Patent Application No. 2021-141210 based on which the present patent application claims priority.

### BRIEF DESCRIPTION OF THE DRAWINGS

FIG. 1 This figure shows chronological gene expression in human chondrocyte progenitor cells differentiated from human iPS cells via human mesodermal cells.

FIG. 2A This figure shows the results of morphological evaluation of spheroids using Cell³ iMager Duos. Bright field images of chondrocyte progenitor spheroids.

FIG. 2B This figure shows the results of morphological evaluation of spheroids using Cell³ iMager Duos. Results of measurement of the diameter of each spheroid with Cell³ iMager Duos.

FIG. 2C This figure shows the results of morphological evaluation of spheroids using Cell³ iMager Duos. Results of measurement of the circularity of each spheroid with Cell³ iMager Duos.

FIG. 2D This figure shows histological staining of human chondrocyte progenitor spheroids.

FIG. 2E This figure shows marker positive ratios based on the histological staining of human chondrocyte progenitor spheroids.

FIG. 3 This figure shows the fusion capacity of human chondrocyte progenitor spheroids.

FIG. 4 This figure shows macro images in vitro of a shaped cartilage.

FIG. 5A This figure shows histological staining in vitro of a shaped cartilage.

FIG. 5B This figure shows marker positive ratios in histological staining in vitro of a shaped cartilage.

FIG. 6 This figure shows gene expression in vitro in a shaped cartilage.

FIG. 7 This figure shows ELISA in vitro of a shaped cartilage.

FIG. 8A This figure shows a macro image of a shaped cartilage after transplantation. Shaped cartilage at the time of resection (arrow heads: contour of the cartilage).

FIG. 8B This figure shows a macro image of a shaped cartilage after transplantation. Shaped cartilage after resection.

FIG. 9 This figure shows CT images of a shaped skeleton after transplantation.

FIG. 10 This figure shows histological images from the immunological staining of a shaped skeleton after transplantation.

FIG. 11 This figure shows macro images in vitro of a shaped cartilage using chondrocyte progenitor cells derived from the auricular perichondrium.

### BEST MODES FOR CARRYING OUT THE INVENTION

Hereinafter, embodiments of the present invention will be described in more detail.

The present invention provides a method for preparing an artificial cartilage tissue, comprising making chondrocyte progenitor cell-containing spheroids into a desired shape while seeding the spheroids on a support; culturing the spheroids while feeding a culture medium from both the obverse and reverse sides of the spheroid-seeded surface to thereby perform fusion among the spheroids; and maturing the resultant fused spheroids into a cartilage tissue in vitro.

Chondrocyte progenitor cells may be obtained by directed differentiation from embryonic stem cells (ES cells) and/or induced pluripotent stem cells (iPS cells). Alternatively, chondrocyte progenitor cells may also be obtained by directed differentiation from perichondrocytes collected from the perichondrium.

Chondrocyte progenitor cells may be induced for differentiation from embryonic stem cells and/or induced pluripotent stem cells. One example of such a method will be explained below. First, embryonic stem cells and/or induced pluripotent stem cells are induced for differentiation into mesodermal cells according to the method described in Cell, Jul. 14, 2016, vol. 166, 451-467. Briefly, additive factors such as Activin, bFGF, Wnt activators (CHIR, WNT3A, etc.) and the like are added to a basal medium which is 1% B 27 and 1% Glutamax-supplemented Dulbecco's Modified Eagle's Medium/Nutrient Mixture F12 (DMEM/F12) while changing the additives every day, and the medium is exchanged daily. At day 5 of directed differentiation, mesodermal cells are obtained. Subsequently, the resultant mesodermal cells are passaged and cultured on plate in Dulbecco's Modified Eagle's Medium/Nutrient Mixture F12 (DMEM/F12) supplemented with TGFβ inhibitors (A8301, SB431542, etc.), PDGFBB, IGF and the like at 36-37° C. with the medium being exchanged every other day. As a result, chondrocyte progenitor cells are obtained in 3 to 5 days. Chondrocyte progenitor cells may be one which expresses SOX9, CD44, CD73 and CD105. The thus obtained chondrocyte progenitor cells may be cultured on plate in Dulbecco's Modified Eagle's Medium/Nutrient Mixture F12 (DMEM/F12) supplemented with TGFβ inhibitors (A8301, SB431542, etc.), PDGFBB, IGF and the like at 36-37° C. with the medium being exchanged 2-4 times a week. Chondrocyte progenitor cells at the passage number of 0-5 may be suitably used.

As embryonic stem cells and/or induced pluripotent stem cells, human-derived cells may be mainly used. However, cells derived from non-human animals (e.g., animals used, for example, as experimental animals, pet animals, working animals, race horses or fighting dogs; more specifically, mouse, rat, rabbit, pig, dog, monkey, cattle, horse, sheep, chicken, shark, ray, coco shark, salmon, shrimp, crab or the like) may also be used.

Chondrocyte progenitor cells may be induced from perichondrocytes by directed differentiation. One example of such a method will be explained below. First, perichondrocytes are collected from the perichondrium present in tissues such as auricular cartilage, costal cartilage, etc. according to the method described in PNAS, Aug. 20, 2011, vol. 108, no. 35, 12279-14484. Briefly, the perichondrium collected from tissues such as auricular cartilage, costal cartilage, etc. is minced and treated with collagenase to thereby isolate perichondrocytes, which are then collected by filtering. The thus obtained perichondrocytes are cultured on plate in Dulbecco's Modified Eagle's Medium/Nutrient Mixture F12 (DMEM/F12) supplemented with TGFβ inhibitors (A8301, SB431542, etc.), PDGFBB, IGF and the like at 36-37° C. with the medium being exchanged every other day. As a result, chondrocyte progenitor cells are obtained in 3 to 5 days. Chondrocyte progenitor cells may be one which expresses SOX9, CD44, CD73 and CD105. The thus obtained chondrocyte progenitor cells may be cultured on plate in Dulbecco's Modified Eagle's Medium/Nutrient Mixture F12 (DMEM/F12) supplemented with TGFβ inhibitors (A8301, SB431542, etc.), PDGFBB, IGF and the like at 36-37° C. with the medium being exchanged 2-4 times a week. Chondrocyte progenitor cells passaged 0 to 5 times may be suitably used.

As perichondrocytes, human-derived cells may be mainly used. However, cells derived from non-human animals (e.g., animals used, for example, as experimental animals, pet animals, working animals, race horses or fighting dogs; more specifically, mouse, rat, rabbit, pig, dog, monkey, cattle, horse, sheep, chicken, shark, ray, coco shark, salmon, shrimp, crab or the like) may also be used.

Chondrocyte progenitor cell-containing spheroids are preferably prepared in a matrix-free manner, that is, without using a surface capable of cell adhesion such as a matrix. For example, such spheroids may be prepared by culturing chondrocyte progenitor cells in a culture vessel with a non-cell-adhesive surface.

The culture vessel with a non-cell-adhesive surface may be one that has received a low adsorption surface treatment. For example, culture vessel whose culture surface is coated with a non-cell-adhesive polymer may be used. Examples of non-cell-adhesive polymer include, but are not limited to, phospholipids, complexes of phospholipid and polymer, poly (2-hydroxyethylmethacrylate) (PHEMA), polyvinyl alcohol, agarose, chitosan, polyethylene glycol, albumin, and photocrosslinkable super-hydrophilic polymers. Examples of culture vessel with a non-cell-adhesive surface include, but are not limited to, Elplasia plate (Corning), Elplasia RB 500 400 NA (Kuraray) and 96-well U bottom plate or V bottom plate (Sumitomo Bakelite). These can be suitably used in the present invention.

The bottom of the culture vessel may suitably have a large number of concavities in a semi-spherical or truncated conical shape. For example, with a 6-well plate having 2885 concavities in a semi-spherical or truncated conical shape (volume of each concavity: 0.068 mm³) per well being used as a culture vessel, 4-5 ml of cell suspension containing 5-7×106 chondrocyte progenitor cells is poured into each well and left to stand in an incubator for 1-5 days. As a result, spheroids 200 μm in size may be formed. Spheroids in microplates may be floated by pipetting, collected in falcon tubes, and subjected to centrifugation so that spheroids will assemble at the bottom of the centrifuge tube. The supernatant may then be sucked to leave spheroids alone.

The medium used for shaping spheroids is not particularly limited and any medium may be used as long as it is capable of shaping spheroids. Preferably, a medium for three-dimensional culture of chondrocyte progenitor cells is preferably used. Specific examples of the medium include, but are not limited to, Dulbecco's Modified Eagle's Medium/Nutrient Mixture F12 (DMEM/F12), Dulbecco's Modified Eagle's Medium (DMEM), F12-Ham, Roswell Park Memorial Institute (RPMI-1640), Eagle's minimum essential medium (EMEM), alpha Modified Eagle Minimum Essential Medium (aMEM), Iscove's Modified Dulbecco's Medium (IMDM) or F-10 Ham, each of which is supplemented with TGFβ (TGFβ1, TGFβ3, etc.), bFGF and Wnt/β catenin inhibitors (Wnt-C59, IWP1, IWP2, IWP3, etc.). In addition to the above additives, antibiotics, antifungal agents, ITS-X, PDGFBB, serum, L-ascorbic acid, dexamethasone, and insulin growth factor may also be added.

Culture may be performed by any method selected from among batch culture, semi-batch culture (fed-batch culture) and continuous culture (perfusion culture). Culture may be either static culture, aeration culture, spinner culture, shaking culture or rotary culture. Among these, static culture is preferable.

The temperature at the time of culture for spheroid formation is preferably 30-40° C., more preferably 37° C.

The time period of culture for spheroid formation is preferably not longer than 5 days, more preferably 1-5 days. The medium may suitably be exchanged once a day.

One spheroid may be composed of 100-7500 cells (preferably 1000-3000 cells). Here, the spheroid-composing cells encompass chondrocyte progenitor cells.

The diameter of spheroid may be 20-1000 μm, preferably 200-350 μm. The circularity of spheroid may suitably be 0.5-1.0, with 0.8-1.0 being preferred. The diameter and the circularity of spheroid may be measured with Cell³ iMager Duos. The positive ratio of SOX9 in spheroid may be 60% or more, preferably 70-100%, and more preferably 80-100%. SOX9 positive ratio of spheroid may be calculated with ImageJ by cutting out the tissue portion from photographed images, conducting separation of three primary colors (red, green and blue), setting a threshold value in the measurement area, and taking the area of SOX9 (green) as numerator, with the area of DAPI (blue) being taken as denominator.

According to the method of the present invention for preparing a cartilage tissue, chondrocyte progenitor cell-containing spheroids are made into a desired shape while seeding the spheroids on a support.

The support may be one through which medium components are capable of passing; which is not toxic to spheroids; and through which spheroids are incapable of passing. If, for example, the support has the structure of a porous membrane, it would be advantageous for culturing spheroids after fusion because nutrients can be fed to fused spheroids from both top and bottom while allowing for efficient oxygen supply. Examples of the support include, but are not limited to, supports whose surface has been negatively charged to acquire hydrophilicity by means of corona discharge in the atmosphere or vacuum gas plasma polymerization treatment (cell adhesion surface treatment) or the like; supports with a gelatin treated surface; supports coated with extracellular matrix (such as collagen, laminin, or fibronectin) or mucopolysaccharides (heparin sulfate, hyaluronic acid, chondroitin sulfate, etc.); supports coated with basic synthetic polymers (such as poly-D-lysine); supports with a synthetic nanofiber surface; supports with a hydrophilic and neutral hydrogel layer's surface; and collagen membrane (KOKEN CO., LTD.). In the case where the support has the structure of a porous membrane, the pore size may be 0.4-8 μm. As a support, Falcon cell culture plate (Corning), Falcon multi-cell culture plate (Corning), Falcon cell culture insert (Corning), or the like may be suitably used.

Spheroids may be seeded on a support using a pipette or spoon. As the desired shape, shapes capable of repairing malformation or damage in cartilage tissues (e.g., auricular cartilage, epiglottic cartilage, costal cartilage, articular cartilage, epiphyseal cartilage, nasal cartilage, tracheal cartilage, pharyngeal cartilage, intervertebral disc, glenoid labrum, meniscus, or symphysis pubis) may be given; as specific examples, rod-like, sheet-like, or spherical shapes may be enumerated. If a more precise or complex shape is required, spheroids may be seeded into a mold prepared in advance and placed on a support, using a pipette or spoon.

Spheroids may be seeded on a support at high density. High density means such that when spheroids that are 150 μm in diameter are taken as an example, the number of spheroids present per cm³ may be 9.5×10⁴-3.8×10⁵, preferably 1.9×10⁵-3.8×10⁵, more preferably 2.9×10⁵-3.8×10⁵.

The number of spheroids may be 2 or more. If the number of spheroids is increased, fused spheroids of larger size can be prepared.

In the method of the present invention, spheroids are fused to each other by making chondrocyte progenitor cell-containing spheroids into a desired shape while seeding the spheroids on a support, and then culturing the spheroids while feeding a culture medium from both the obverse and reverse sides of the spheroid-seeded surface.

For example, chondrocyte progenitor cell-containing spheroids are seeded in an intended shape on the membrane of a cell culture insert. Then, a culture medium is added to the lower part of the membrane so that the spheroids on the membrane will be immersed in the medium in culture plates. Fusion among spheroids can be achieved by leaving the culture plates to stand in an incubator.

The term “fusion among spheroids” means that a plurality of spheroids form a continuous structure, in which disappearance of the contour of each spheroid is confirmed. Fusion among spheroids may achieve the following: the size of spheroid is increased; and a cartilage tissue of large size can be prepared by maturing fused spheroids (that is, inducing chondrocyte progenitor cells contained in the fused spheroids to differentiate into chondrocytes).

The medium to be used for fusion among spheroids is not particularly limited and any medium may be used as long as it is suitable for fusion among spheroids. Preferably, a medium for the above-described three-dimensional culture of chondrocyte progenitor cells may be used. Specific examples of the medium include, but are not limited to, Dulbecco's Modified Eagle's Medium/Nutrient Mixture F12 (DMEM/F12), Dulbecco's Modified Eagle's Medium (DMEM), F12-Ham, Roswell Park Memorial Institute (RPMI-1640), Eagle's minimum essential medium (EMEM), alpha Modified Eagle Minimum Essential Medium (aMEM), Iscove's Modified Dulbecco's Medium (IMDM) or F-10 Ham, each of which is supplemented with TGF (TGFβ1, TGFβ3, etc.), bFGF and Wnt/B catenin inhibitors (Wnt-C59, IWP1, IWP2, IWP3, etc.). In addition to the above additives, antibiotics, antifungal agents, ITS-X, PDGFBB, serum, L-ascorbic acid, dexamethasone, and insulin growth factor may also be added.

Culture may be either static culture or shaking culture, with static culture being preferable.

The temperature at the time of culture for fusion among spheroids is preferably 30-40° C., more preferably 37° C.

The time period of culture for fusion among spheroids is preferably 12 hours to 4 days, more preferably 12 hours to 1 day. It is appropriate to exchange the medium every two days.

Upon confirmation of fusion among spheroids, fused spheroids are matured into a cartilage tissue in vitro. For maturation of the fused spheroid into a cartilage tissue, culture may be continued after the medium fed from both the obverse and reverse sides of the spheroid-seeded surface is changed to a cartilage differentiation medium. As the cartilage differentiation medium, any medium may be used as long as it is capable of maturing fused spheroids into a cartilage tissue. For example, Dulbecco's Modified Eagle's Medium/Nutrient Mixture F12 (DMEM/F12), Dulbecco's Modified Eagle's Medium (DMEM), F12-Ham, Roswell Park Memorial Institute (RPMI-1640), Eagle's minimum essential medium (EMEM), alpha Modified Eagle Minimum Essential Medium (aMEM), Iscove's Modified Dulbecco's Medium (IMDM) or F-10 Ham, each of which is supplemented with BMP (BMP4, BMP2, etc.) may be used. Other additives such as antibiotics, antifungal agents, ITS-X, TGFβ (TGFβ1, TGFβ3, etc.), bFGF, PDGFBB, Wnt/β catenin inhibitors (Wnt-C59, IWP1, IWP2, IWP3, etc.), serum, L-ascorbic acid, dexamethasone and insulin growth factor may also be added. Further, L-proline may also be added.

Maturing into cartilage may be confirmed by HE staining, Alcian blue staining, or immune-histological staining (type II collagen and type I collagen).

A cartilage tissue obtained by maturing fused spheroids has the following characteristics: hardness is improved; type II collagen (immunohistological marker for cartilage tissue) is positive extensively: type I collagen (marker for perichondrium) is positive mainly in the peripheral area of the cartilage tissue: and gene expression of SOX9 (chondrocyte progenitor marker) and COL11A2 (cartilage marker) can be raised. In immunohistological staining, the positive ratio of type II collagen in cartilage tissue may be 60% or more, preferably 70-90%, and the positive ratio of type I collagen may be 20% or less, preferably 5-15%. The positive ratios of type II and type I collagens, respectively, in cartilage tissue can be calculated with ImageJ by cutting out the tissue portion from photographed images, conducting separation of three primary colors (red, green and blue), setting a threshold value in the measurement area, and taking the areas of red (type II collagen) and green (type I collagen) as numerators, with the area of blue taken as denominator.

According to the method of the present invention, it is possible to prepare an artificial cartilage tissue with a diameter (φ) of 2 mm or more, 6 mm or more, 40 mm or more, or 80 mm or more and a thickness of 0.5 mm or more, 1 mm or more, 5 mm or more, or 15 mm or more. The present invention provides an artificial cartilage tissue prepared by the above-described method, which is 6 mm or more in diameter and 0.5 mm or more in thickness. The artificial cartilage tissue which is 6 mm or more in diameter and 0.5 mm or more in thickness may be prepared from 10-10000 spheroids sized 20-500 μm in diameter.

It is possible to prepare an artificial cartilage tissue which is 0.5-15 mm in diameter and 2-80 mm in length from 100-30000 spheroids which are 20-1000 μm in diameter. It is possible to prepare an artificial cartilage tissue which is 2-5 mm in diameter and 4-40 mm in length from 500 -1500 spheroids which are 200-350 μm in diameter.

The hardness of the artificial cartilage tissue is appropriately 0.2-1.0 MPa, preferably 0.4-0.6 MPa. The hardness of the artificial cartilage tissue may be measured with a tabletop tester (Shimadzu Corporation: EZ-Test EZ-SX Jig S346-57829-02).

A cartilage tissue matured in vitro may be transplanted into a non-human animal and matured further. A cartilage tissue matured in vitro may also be transplanted into a non-human animal and matured into a bone tissue. As exemplary non-human animals, mouse, rat, monkey and pig may be enumerated. When a cartilage tissue obtained by culturing fused spheroids for a short period of time (a shaped cartilage) is transplanted into a living body, a more matured cartilage may be obtained. When a cartilage tissue obtained by culturing fused spheroids for a long period of time (a shaped hypertrophic cartilage) is transplanted into a living body, a bone tissue may be obtained. In a shaped hypertrophic cartilage, hypertrophic chondrocytes with hypertrophied cytoplasm are observed. Generally, hypertrophic chondrocytes may be observed in immunohistochemical staining with type 10 collagen used as a marker.

The temperature at the time of culture of fused spheroids for maturing into a shaped cartilage is preferably 30-40° C., more preferably 37° C.

The time period of culture of fused spheroids for maturing into a shaped cartilage is preferably 14-42 days, more preferably 21-28 days. The medium may suitably be exchanged every 2-3 days. When a shaped cartilage is subcutaneously transplanted, the cartilage may become a more matured cartilage. The time period of transplantation may be 14-182 days, preferably 28-56 days. Maturation of cartilage may be confirmed by cartilage cavity in HE staining and disappearance of type I collagen in immunohistological staining.

The temperature at the time of culture of fused spheroids for maturing into a shaped hypertrophic cartilage is preferably 30-40° C., more preferably 37° C.

The time period of culture of fused spheroids for maturing into a shaped hypertrophic cartilage is preferably 42-84 days, more preferably 56-70 days. The medium may suitably be exchanged every 2-3 days. For maturation into bone tissue and osteochondral transitional zone, a shaped hypertrophic cartilage may be subcutaneously transplanted. The time period of transplantation of the shaped hypertrophic cartilage for maturing into a bone tissue may be 28 days or more, preferably 56 days or more. The resultant bone tissue may be confirmed by CT images and histological staining and the resultant osteochondral transitional zone may be confirmed by histological staining. The diameter of shaped hypertrophic cartilage may be 1-20 mm, preferably 5-10 mm. The thickness of shaped hypertrophic cartilage may be 2-100 mm, preferably 20-40 mm. The present invention also provides an artificial cartilage tissue prepared by the above-described method, wherein a part or the whole of the cartilage tissue is differentiated into bone tissue after transplantation into a living body (that is, a shaped hypertrophic cartilage is provided).

The present invention also provides a method for preparing an artificial bone tissue, comprising transplanting an artificial cartilage tissue prepared by the above-described method into a non-human animal and maturing the cartilage tissue into a bone tissue. According to the method of the present invention, it is possible to prepare an artificial bone tissue with a diameter (φ) of 2 mm or more, 6 mm or more, 40 mm or more, 6 mm or more, or 80 mm or more and a thickness of 0.5 mm or more, 1 mm or more, 5 mm or more, or 15 mm or more. The present invention also provides an artificial bone tissue prepared by the above-described method, which is 6 mm or more in diameter and 0.5 mm or more in thickness.

An artificial cartilage tissue (which may or may not have been transplanted into a living body) and/or an artificial bone tissue prepared by the method of the present invention is applicable to treatment of achondroplasia, etc. in the craniofacial area, treatment of osteoarthritis, etc., and other aspects of regenerative medicine where shapes are considered important. The present invention provides a composition comprising an artificial cartilage tissue prepared by the method of the present invention, which is used for transplantation into a living body to compensate for a deficiency of cartilage tissue and/or bone tissue therein. When the artificial cartilage tissue is a shaped cartilage, it is possible to transplant the artificial cartilage tissue into a living body to compensate for a deficiency of cartilage tissue therein. When the artificial cartilage tissue is a shaped hypertrophic cartilage, it is possible to transplant the artificial cartilage tissue into a living body to compensate for a deficiency of bone tissue therein. The present invention also provides a composition comprising an artificial bone tissue prepared by the above-described method, which is used for transplantation into a living body to compensate for a deficiency of bone tissue therein.

Specifically, the artificial cartilage tissue prepared by the method of the present invention is transplanted into areas of hypoplastic cartilage tissue (such as saddle nose or microtia) for treatment. Furthermore, deformed auricles or noses due to traffic or sport injuries can be treated according to the method of the present invention by transplanting a cartilage tissue that reproduces a more complex morphology. What is more, osteoarthritis or the like can be treated by transplanting the artificial cartilage tissue as prepared by the method of the present invention into cartilage defects on articular surfaces. The artificial bone tissue of the present invention is applicable to “transplantation into facial bone defects due to trauma”, “cosmetic surgery such as transplantation into the nasal bone area to raise the nose bridge, transplantation into the cheekbone area to raise the cheekbones, or transplantation into the mandible bone area to form jaw lines”, “bone transplantation into pseudarthrosis area to cope with bone nonunion after fracture”, or “bone transplantation to cope with bone defects that occur at the time of removal of tumor such as osteosarcoma”.

### EXAMPLES

Hereinbelow, the present invention will be described in more detail with reference to the following Example.

### Example 1

### Culture of Human iPS Cells

AK02 medium (Ajinomoto) (1.5 ml) containing 7 μl of iMatris-511 (Nippi) and 1.5 μl of Y-27632 (FUJIFILM Wako Pure Chemical) was added to each well of 6-well plates, which were then left to stand in an incubator at 37° C. for 1 hour. Human iPS cells (Center for iPS Cell Research and Application, Kyoto University: 1383D6) (5×10³) were seeded in each well of the plates. Medium exchange with 1.5 ml of AK 02 medium (Ajinomoto) was carried out every day and a plurality of colonies were confirmed at day 7. At the time of passaging, human iPS cells were washed with PBS, 500 μl of Accutase (ICT) solution was added, and the resultant cells were left to stand in an incubator at 37° C. for 6 minutes. After peeling off the cells by pipetting, 5 ml of AK02 medium (Ajinomoto) was added, and centrifugation was conducted at 900 rpm for 5 minutes. Then, the cells were suspended in AK02 medium (Ajinomoto) and subjected to further culturing of iPS cells or directed differentiation into chondrocyte progenitor cells.

Directed Differentiation From Human iPSC Cells Into Human Mesodermal Cells

AK02 medium (Ajinomoto) (1.5 ml) containing 7 μl of iMatris-511 (Nippi) and 1.5 μl of Y-27632 was added to each well of 6-well plates, which were then left to stand in an incubator at 37° C. for 1 hour. Human iPS cells were seeded at a density of 1-1.5×10⁵ cells/well. On the next day, the medium was exchanged with DMEM/F12 Ham (1:1) (Sigma-Aldrich) supplemented with 1% Glutamax, 1% B27, 4 μM CHIR (CAYMAN), 100 nM PIK90 (EMD Millipore), 30 ng/ml Activin and 20 ng/ml bFGF. On the next day, the medium was exchanged with DMEM/F12 Ham (1:1) (Sigma-Aldrich) supplemented with 1% Glutamax, 1% B27, 3 μM CHIR (CAYMAN), 250 nM DMH1 (Selleck) and 20 ng/ml bFGF (Wako). On the next day, the medium was exchanged with DMEM/F12 Ham (1:1) (Sigma-Aldrich) supplemented with 1% Glutamax, 1% B27, 1 μM A8301 (TOCRIS), 250 nM DMHI (Selleck), 250 nM PD0325901 (TOCRIS) and 1 μM C59 (Cellagen Tech). On the next day, the medium was exchanged with DMEM/F12 Ham (1:1) (Sigma-Aldrich) supplemented with 1% Glutamax, 1% B27, 1 μM C59 (Cellagen Tech) and 5 nM SAG21K (TOCRIS), and cells were cultured therein for 2 days. Then, gene expression analysis was conducted using markers for human mesodermal cells (such as HOXB5, FOXF1, etc.).

### Collection of Human Auricular Perichondrocytes

Human perichondrium samples (perichondrium samples obtained from surplus human auricular cartilage arising from surgery were used with the consent of patients or their parents. Approval obtained from the respective Ethics Committees of Kanagawa Children's Medical Center and Yokohama City University Hospital.) were minced with scissors until no solid mass was left. The minced sample was shaken in 0.2% collagenase solution (Worthington) at 37° C. and 600 rpm for 2 hours to thereby isolate human perichondrocytes. The resultant suspension was filtered with a 40-um cell strainer and centrifuged at 1500 rpm for 5 minutes to collect human perichondrocytes.

### Directed Differentiation Into Human Chondrocyte Progenitor Cells

Seven milliliters of 0.1% gelatin was added to each of 10 cm dishes, which were then left to stand in an incubator at 37° C. for 1 hour. After removal of the supernatant from the dishes, 8 ml of DMEM/F12 Ham (1:1) (Sigma-Aldrich) supplemented with 1% Antibiotic Antimycotic Solution (Sigma-Aldrich), 1% ITS-X (Gibco™M), 1 μM A8301 (TOCRIS), 20 ng/ml bFGF (Wako), 30 ng/ml PDGFBB (Peprotech), 1 μM WntC59 (Cellagen Tech), 4% Fetal Bovine Serum (Biowest), 40 μg/ml L-ascorbic acid (Sigma-Aldrich), 40 μg/ml dexamethasone (Sigma-Aldrich) and 10 ng/ml Insulin Growth Factor (Sigma-Aldrich) was added to the dishes. Human mesodermal cells or human auricular perichondrocytes (1.2×10⁶) were seeded in the dishes, which were then left to stand in an incubator at 37° C. After 48 hours, medium exchange was carried out. Twenty-four hours later, the dishes were confirmed to have become confluent, and gene expression analysis was conducted using markers for human chondrocyte progenitor cells (SOX9, CD44, CD73, CD105)

### Formation of Human Chondrocyte Progenitor Spheroids by Three-Dimensional Culture of Human Chondrocyte Progenitor Cells

Chondrocyte progenitor cells (derived from either human iPS cells or human auricular cartilage) seeded in 10-cm dishes were washed with PBS and peeled off by treatment with a trypsin solution for 3 minutes. The trypsin solution containing chondrocyte progenitor cells was deactivated with 10% fetal bovine serum (Biowest)-containing DMEM/F12 in a volume equivalent to three volumes of the trypsin solution, and collected into falcon tubes. The resultant trypsin solution was centrifuged under 400 G for 3 minutes. After removal of the supernatant, cells were suspended in DMEM/F12 Ham (1:1) (Sigma-Aldrich) supplemented with 1% Antibiotic Antimycotic Solution (Sigma-Aldrich), 1% ITS-X (Gibco™M), 15 ng/ml TGFβ1 (Peprotech), 15 ng/ml bFGF (Wako), 10 ng/ml PDGFBB (Peprotech), 1 μM WntC59 (Cellagen Tech), 4% Fetal Bovine Serum (Biowest), 40 μg/ml L-ascorbic acid (Sigma-Aldrich), 40 μg/ml dexamethasone (Sigma-Aldrich) and10 ng/ml Insulin Growth Factor (Sigma-Aldrich) to give a density of 1.4×106 cells/ml. Five milliliters of this chondrocyte progenitor cell suspension (containing 7×10°chondrocyte progenitor cells) was poured into each well of Elplasia plates (Corning: 6- well standard), which were then left to stand in an incubator at 37° C. On the next day, 4 ml medium exchange was conducted, and cells were cultured further for 24 hours.

### Induction From Human Chondrocyte Progenitor Spheroids to Human Shaped Cartilage

Chondrocyte progenitor spheroids in wells of Elplasia plates (Corning: 6-well standard) were transferred into falcon tubes by pipetting. After centrifugation at 1000 rpm for 2 minutes, the supernatant was removed. The remaining chondrocyte progenitor spheroids were collected with a pipette and seeded on the membrane of each well of cell culture insert with 0.4 μm pores (Falcon: 6-well standard) in such a manner that the spheroids would be made into an intended shape. To the lower part of the membrane, 3 ml of DMEM/F12 Ham (1:1) (Sigma-Aldrich) supplemented with 1% Antibiotic Antimycotic Solution (Sigma-Aldrich), 1% ITS-X (GibcoTM), 15 ng/ml TGFβ1 (Peprotech), 15 ng/ml bFGF (Wako), 10 ng/ml PDGFBB (Peprotech), 1 μM WntC59 (Cellagen Tech), 4% Fetal Bovine Serum (Biowest), 40 μg/ml L-ascorbic acid (Sigma-Aldrich), 40 μg/ml dexamethasone (Sigma-Aldrich) and 10 ng/ml Insulin Growth Factor (Sigma-Aldrich) was added. Then, the culture insert was left to stand in an incubator at 37° C. Hereinbelow, a shaped cartilage was prepared in the form of a rod. The seeded chondrocyte progenitor spheroids were fused with each other in about 12 hours, and disappearance of the contour of each spheroid could be confirmed under the microscope. On the next day, the medium in the lower part of the membrane was exchanged in the entire volume (3 ml). After three days, the medium was changed to DMEM/F12 Ham (1:1) (Sigma-Aldrich) supplemented with 1% Antibiotic Antimycotic Solution (Sigma-Aldrich), 1% ITS-X (Gibco™), 5 ng/ml TGFβ1 (Peprotech), 10 ng/ml bFGF (Wako), 5 ng/ml PDGFBB (Peprotech), 20 ng/ml BMP 4, 1 μM WntC59 (Cellagen Tech), 2% Fetal Bovine Serum (Biowest), 40 μg/ml L-ascorbic acid (Sigma-Aldrich), 40 μg/ml dexamethasone (Sigma-Aldrich) and 10 ng/ml Insulin Growth Factor (Sigma-Aldrich). After another three days, the medium was changed to DMEM/F12 Ham (1:1) (Sigma-Aldrich) supplemented with 1% Antibiotic Antimycotic Solution (Sigma-Aldrich), 1% ITS-X (Gibco™), 2.5 ng/ml TGFβ1 (Peprotech), 1 ng/ml bFGF (Wako), 20 ng/ml BMP 4 (R & D), 1% Fetal Bovine Serum (Biowest), 40 μg/ml L-ascorbic acid (Sigma-Aldrich), 40 μg/ml dexamethasone (Sigma-Aldrich), 10 ng/ml Insulin Growth Factor (Sigma-Aldrich) and 40 μg/ml L-proline (Sigma-Aldrich). After another three days, the medium was changed to DMEM/F12 Ham (1:1) (Sigma-Aldrich) supplemented with 1% Antibiotic Antimycotic Solution (Sigma-Aldrich), 1% ITS-X (Gibco™), 10 ng/ml BMP 4, 0.5% Fetal Bovine Serum (Biowest), 40 μg/ml L-ascorbic acid (Sigma-Aldrich), 40 μg/ml dexamethasone (Sigma-Aldrich), 10 ng/ml Insulin Growth Factor (Sigma-Aldrich) and 40 μg/ml L-proline (Sigma-Aldrich). From that time on, the medium was exchanged in every 3 days. According to the above-described procedures, a cartilage tissue expressing type II collagen and Alcian blue was obtained by culturing for 20 days after the seeding on the membrane.

### Induction From Human Chondrocyte Progenitor Spheroids to Human Shaped Hypertrophic Cartilage

Chondrocyte progenitor spheroids in wells of Elplasia plates (Corning: 6-well standard) were transferred into falcon tubes by pipetting. After centrifugation at 1000 rpm for 2 minutes, the supernatant was removed. The remaining chondrocyte progenitor spheroids were collected with a pipette and seeded on the membrane of each well of cell culture insert with 0.4 μm pores (Falcon: 6-well standard) in such a manner that the spheroids would be made into an intended shape. To the lower part of the membrane, 3 ml of DMEM/F12 Ham (1:1) (Sigma-Aldrich) supplemented with 1% Antibiotic Antimycotic Solution (Sigma-Aldrich), 1% ITS-X (Gibco™), 15 ng/ml TGFβ1 (Peprotech), 15 ng/ml bFGF (Wako), 10 ng/ml PDGFBB (Peprotech), 1 μM WntC59 (Cellagen Tech), 4% Fetal Bovine Serum (Biowest), 40 μg/ml L-ascorbic acid (Sigma-Aldrich), 40 μg/ml dexamethasone (Sigma-Aldrich) and 10 ng/ml Insulin Growth Factor (Sigma-Aldrich) was added. Then, the culture insert was left to stand in an incubator at 37° C. Hereinbelow, a shaped cartilage was prepared in the form of a rod, namely a cartilage mimicking the shape of nose or ear. The seeded chondrocyte progenitor spheroids fused with each other in about 12 hours, and disappearance of the contour of each spheroid could be confirmed under the microscope. On the next day, the medium in the lower part of the membrane was exchanged in the entire volume (3 ml). After three days, the medium was changed to DMEM/F12 Ham (1:1) (Sigma-Aldrich) supplemented with 1% Antibiotic Antimycotic Solution (Sigma-Aldrich), 1% ITS-X (Gibco™), 5 ng/ml TGFβ1 (Peprotech), 10 ng/ml bFGF (Wako), 5 ng/ml PDGFBB (Peprotech), 20 ng/ml BMP 4, 1 μM WntC59 (Cellagen Tech), 2% Fetal Bovine Serum (Biowest), 40 μg/ml L-ascorbic acid (Sigma-Aldrich), 40 μg/ml dexamethasone (Sigma-Aldrich) and 10 ng/ml Insulin Growth Factor (Sigma-Aldrich). After another three days, the medium was changed to DMEM/F12 Ham (1:1) (Sigma-Aldrich) supplemented with 1% Antibiotic Antimycotic Solution (Sigma-Aldrich), 1% ITS-X (Gibco™), 2.5 ng/ml TGFβ1 (Peprotech), 1 ng/ml bFGF(Wako), 20 ng/ml BMP 4 (R & D), 1% Fetal Bovine Serum (Biowest), 40 μg/ml L-ascorbic acid (Sigma-Aldrich), 40 μg/ml dexamethasone (Sigma-Aldrich), 10 ng/ml Insulin Growth Factor (Sigma-Aldrich) and 40 μg/ml L-proline (Sigma-Aldrich). After another three days, the medium was changed to DMEM/F12 Ham (1:1) (Sigma-Aldrich) supplemented with 1% Antibiotic Antimycotic Solution (Sigma-Aldrich), 1% ITS-X (Gibco™), 10 ng/ml BMP 4, 0.5% Fetal Bovine Serum (Biowest), 40μg/ml L-ascorbic acid (Sigma-Aldrich), 40 μg/ml dexamethasone (Sigma-Aldrich), 10 ng/ml Insulin Growth Factor (Sigma-Aldrich) and 40 μg/ml L-proline (Sigma-Aldrich). From that time on, the medium was exchanged in every 3 days. According to the above-described procedures, a hypertrophic cartilage characterized by type II collagen-positive and Alcian blue-positive extracellular matrix and hypertrophic chondrocytes with hypertrophied cytoplasm was obtained by culturing for 50 days after the seeding on the membrane.

### Transplantation and Resection of Shaped Cartilage

Mice (NOD/SCID) or rats (IL2rg-KO) were anesthetized by isoflurane (Pfizer: 1 ml/1 ml) inhalation. The hair at the site of transplantation was removed. Depending on transplantation samples, skin incision was performed with scissors and toothed forceps. A sample cultured three-dimensionally for 15-30 days was subcutaneously implanted and the incision was closed by suturing at intervals of 2 mm with No. 6-0 surgical needle. At the time of resection, anesthetization by isoflurane inhalation was also conducted and the transplanted sample was resected with scissors and toothed forceps. Depending on the analysis to be conducted, the sample was preserved in PBS or formalin solution.

### Induction From Shaped Hypertrophic Cartilage to Shaped Skeleton

Mice (NOD/SCID) or rats (IL2rg-KO) were anesthetized by isoflurane (Pfizer: 1 ml/1 ml) inhalation. The hair at the site of transplantation was removed. Depending on transplantation samples, skin incision was performed with scissors and toothed forceps. A sample cultured three-dimensionally for 56-70 days was subcutaneously implanted and the incision was closed by suturing at intervals of 2 mm with No. 6-0 surgical needle. Ossification was confirmed by micro-CT in samples one month and two months after transplantation. At the time of resection, anesthetization by isoflurane inhalation was also conducted and the transplanted sample was resected with scissors and toothed forceps. Depending on the analysis to be conducted, the sample was preserved in PBS or formalin solution.

### Gene Expression Analysis

The chondrocyte progenitor cells obtained in 10-cm dishes were collected and RNA was purified using PureLink RNA mini kit (Thermo Fisher Scientific). For cDNA synthesis, High Capacity cDNA Reverse Transcription Kit (Thermo Fisher Scientific) was used. For quantitative determination of genes, 18S rRNA (Applied Biosystems) was used as an internal standard. Genes were amplified/detected using Light Cycler™ 480 (Roche Life Science).

### High-Precision Bright Field Analysis of Chondrocyte Progenitor Spheroids

Elplasia plates were left to stand in Cell3 iMager Duos (SCREEN), high-precision bright field analysis was conducted, and the circularity (0-1.0) and the diameter (um) of each spheroid were measured automatically.

### Evaluation of Fusion of Chondrocyte Progenitor Spheroids

Immediately after, 12 hours after and 10 days after seeding chondrocyte progenitor spheroids in cell culture inserts, the peripheryof shaped cartilage was confirmed using a microscope (Olympus 1X 73) under magnification 20×.

### Hardness Measurement and Analysis of Shaped Cartilage Tissue

The hardness of shaped cartilage tissue was measured using a tabletop tester (Shimadzu Corporation: EZ-Test EZ-SX Jig S 346-57829-02). With the plunger pushed in at 3 mm/min, elastic modulus (MPa) at 0.2 mm-0.6 mm was determined.

### Functional Analysis of Shaped Cartilage Tissue With Elisa

Capture, Detection and Standard for DuoSet™ (R&D) were prepared from PBS solution according to the specifications described in the Certificate of Analysis. Capture solution was added to 96-well plates at 100 μl/well. The plates were wrapped in plastic wrap and left to stand at room temperature overnight. After removal of Capture solution from each well, PBS-Tween solution was added to the plate at 200 μl/well. This operation was repeated three times. The plate was put on Kim towel to wipe off the moisture content. Block Ace (DC Pharma) solution was added to the plate at 300 μl/well. The plate was wrapped in plastic wrap and left to stand at room temperature for 1 hour. After removal of the solution from the plate, PBS-Tween solution was added to the plate at 200 μl/well. This operation was repeated three times. The plate was put on Kim towel to wipe off the moisture content. Standard, Sample and Blank were added to respective wells in proportions of 100 μl. The plate was wrapped in plastic wrap and left to stand at room temperature for 2 hours. After removal of the solution from the plate, PBS-Tween solution was added to the plate at 200 μl/well. This operation was repeated three times. The plate was put on Kim towel to wipe off the moisture content. Then, Detection solution was added at 100 μl/well. After removal of the solution from the plate, PBS-Tween solution was added to the plate at 200 μl/well. This operation was repeated three times. The plate was put on Kim towel to wipe off the moisture content. Streptavidin solution (light shielding required) was prepared in the amount required to give a concentration specified in the Certificate of Analysis as WORKING CONCENTRATION, and the solution was added to the plate at 100 μl/well. The plate was wrapped in aluminum foil and left to stand at room temperature for 20 minutes. After removal of the solution from the plate, PBS-Tween solution was added to the plate at 200 μl/well. This operation was repeated three times. The plate was put on Kim towel to wipe off the moisture content. Then, TMB one Solution (light shielding required) was added at 50 μl/well. The plate was wrapped in aluminum foil and observation was made for several minutes until color development occurred at room temperature. After confirmation of color development, HCL was added at 50 μl/well. Absorbance at 450 nm was measured with a plate reader. As a reference, absorbance at 540 nm or 570 nm was also measured. A calibration curve was drawn to calculate concentrations.

### Preparation of Paraffin Sections

The collected samples were left to stand in formalin (FUJIFILM Wako Pure Chemical) solution overnight for fixation. The fixed sample was washed with PBS solution at room temperature for 30 minutes and with 70% ethanol at room temperature for 30 minutes. Using an automatic embedding machine, the resultant sample was dehydrated with 100% ethanol for 1 hour (7 times). Subsequently, the sample was immersed in xylene for 1 hour (3 times) and 100% paraffin for 1 hour (4 times) to effect embedding. The embedded sample was sliced into 2-4 μm thick sections with a microtome. After slicing, the sample was dried at 42° C.

### Hematoxylin-eosin Staining

Paraffin sections were deparaffinized and hydrophilized using xylene, 100% ethanol, 95% ethanol, 90% ethanol, 70% ethanol and Milli-Q™ water in this order. The resultant sections were stained in hematoxylin solution (Muto Pure Chemicals) for 20 minutes. After washing with running water for 10 minutes, sections were stained in eosin solution (Muto Pure Chemicals) for 2 minutes. After washing with running water, sections were dehydrated using ethanol at gradually varying concentrations and cleared with xylene solution. Subsequently, a water-insoluble mounting medium was placed on the sample, which was covered with a cover glass for inclusion.

### Alcian Blue Staining

Paraffin sections were deparaffinized and hydrophilized using xylene, 100% ethanol, 95% ethanol, 90% ethanol, 70% ethanol and Milli-Q™ water in this order. After treating in 3% aqueous acetic acid solution for 1 minute, sections were stained in Alcian blue solution (pH 2.5) (Wako) for 40 minutes. After treating in 3% aqueous acetic acid solution for 5 minutes, sections were washed with running water for 5 minutes. Subsequently, sections were stained in Kernechtrot solution (Muto Pure Chemicals) for 5 minutes and washed with running water for 1 minute. The resultant sections were dehydrated using ethanol in gradually varying concentrations and cleared with xylene solution. Finally, a water-insoluble mounting medium was placed on the section, which was covered with a cover glass for inclusion.

### Immunohistochemical Analysis

Paraffin sections were deparaffinized and hydrophilized using xylene, 100% ethanol, 95% ethanol, 90% ethanol, 70% ethanol and Milli-Q™ water in this order. After washing samples with 0.1% TBS-Tween solution for 5 minutes twice, pepsin (Abcam) was added thereto, and activation of antigen was conducted at room temperature for 20 minutes. After washing with 1% TBS-Tween solution for 5 minutes, protein blocker (Dako) treatment was conducted at room temperature for 30 minutes. Subsequently, samples were reacted with diluted primary antibody (Anti-Collagen Type II Antibody, clone 6B3 (Merck), or Anti-Collagen type I, Human, rabbit-polyclonal (ACRIS)) at room temperature for 2 hours or at 4° C. overnight. After washing with 1% TBS-Tween solution for 5 minutes 3 times, samples were reacted with fluorescence-labeled secondary antibody at room temperature for 1 hour. After washing with 1% TBS-Tween solution for 5 minutes 3 times, samples were subjected to inclusion using Apathy's Mounting Media with DAPI and a cover glass for microscopic observation. The positive ratio of each antibody was calculated with Image J as a ratio (%) of positive area of each antibody to DAPI positive area.

### Experimental Results

Induction for Differentiation from Human iPS Cells to Human Mesodermal Cells, and from Human Mesodermal Cells to Human Chondrocyte Progenitor Cells

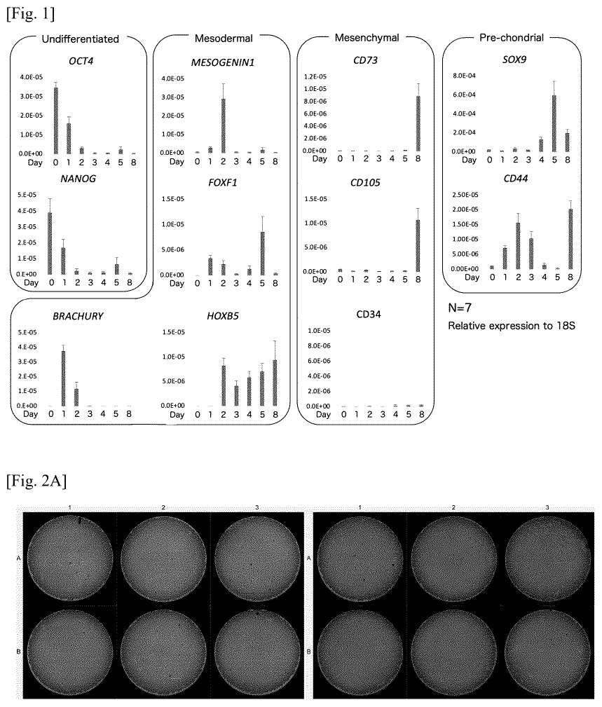

Directed differentiation from human iPS cells (day 0) to human chondrocyte progenitor cells (day 8) via human mesodermal cells (day 5) was conducted, and gene expression was confirmed daily (FIG. 1). Undifferentiated markers OCT4 and NANOG were highly expressed at day 0, and a decrease in their expression was observed over time. As for human mesodermal markers, the expressions of BRACHURY, MESOGININ1, FOXF1 and HOXB5 (in this order from the early period) were shown to increase over time. These results were consistent with the embryological rising order. Further, at day 8, chondrocyte progenitor cells were obtained (n=7) that featured high expression of pre-chondrial markers SOX9 and CD44 and which also expressed mesenchymal markers CD73 and CD105 (FIG. 1).

High-Precision Bright Field Evaluation of Chondrocyte Progenitor Spheroids from Human Chondrocyte Progenitor Cells

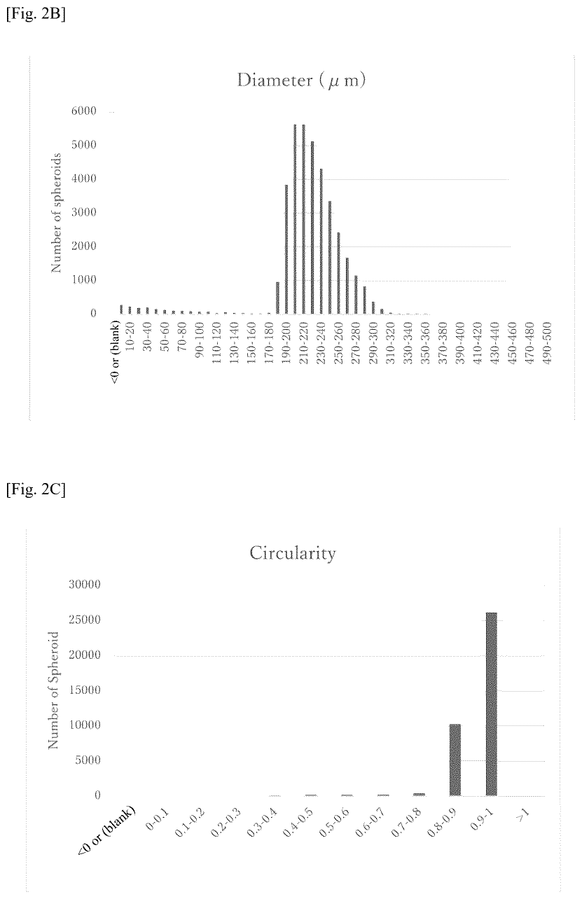

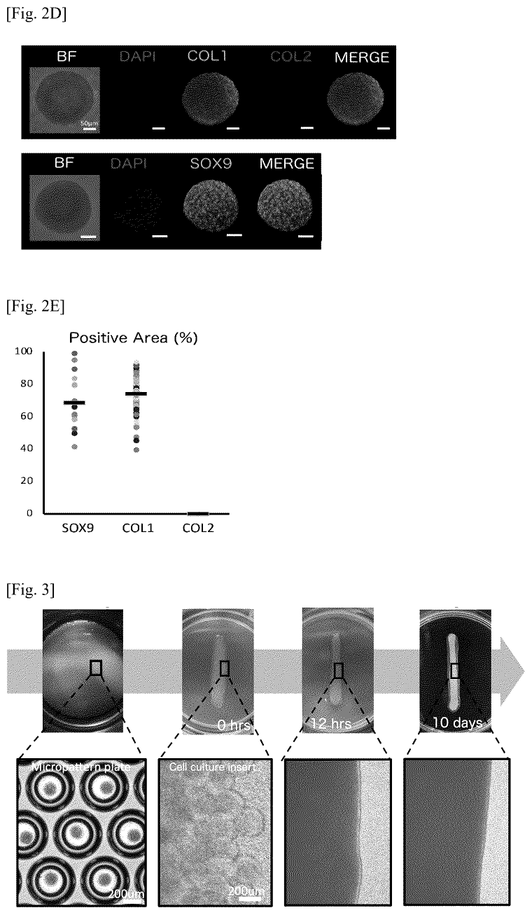

A large number of spheroids were formed in 48 hours after seeding of chondrocyte progenitor cells in Elplasia plates (FIG. 2A). The diameter and the circularity of each spheroid were measured with Cell 3 iMager Duos. Of the 37344 spheroids, 34261 (91.7%) were 200-300 μm in diameter, and 36262 (97.1%) were 0.8-1.0 in circularity (FIGS. 2B and 2C).

In vitro Histological Analysis of Human Chondrocyte Progenitor Spheroids

By immunohistological staining, human chondrocyte progenitor spheroids were confirmed to be positive for pre-chondrial marker SOX9 and for type I collagen (FIG. 2D). SOX9-positive ratio and type I collagen-positive ratio within spheroids were 69% and 74%, respectively (FIG. 2E).

### Fusion Capacity of Human Chondrocyte Progenitor Spheroids

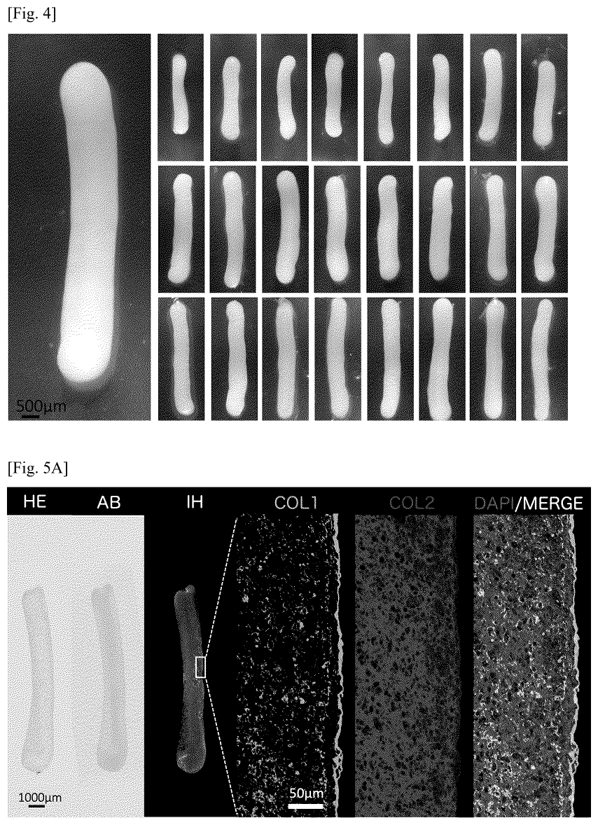

Spheroids prepared in Elplasia plates were seeded on cell culture inserts in a desired shape (rod in this Example). At the time of seeding, spheroids approx. 200 μm in size were confirmed on cell culture inserts under microscope but 12 hours later, the borderline of each spheroid disappeared and fusion among spheroids was confirmed (FIG. 3).

In vitro Histological Analysis of Shaped Cartilage

As a result of three-dimensional culture in cell culture inserts for 20 days, a shaped cartilage was obtained with high reproducibility in macro images (n=24) (FIG. 4). The resultant shaped cartilage was stainable with Alcian blue which would stain the extracellular matrix of cartilage tissue; and type II collagen (cartilage tissue marker) was positive extensively (about 80% positive area). Further, type I collagen, a perichondrium marker, was also positive centering on the periphery of shaped cartilage, with positive area being about 5% (n=4) (FIGS. 5A, 5B).

In vitro Functional Analysis of Shaped Cartilage

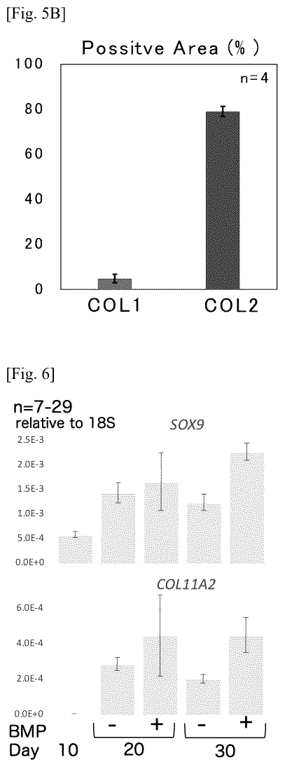

Quantitative RT-PCR revealed that expressions of pre-chondrial marker SOX9 and cartilage marker COL11A2 increased time-dependently in chondrocyte progenitor spheroids (at day 10 of culture), shaped cartilage (at day 20 of culture) and shaped cartilage (at day 30 of culture) (n=7−29) (FIG. 6). Upon BMP administration, these markers showed still higher gene expression than when no BMP was administered(FIG. 6).

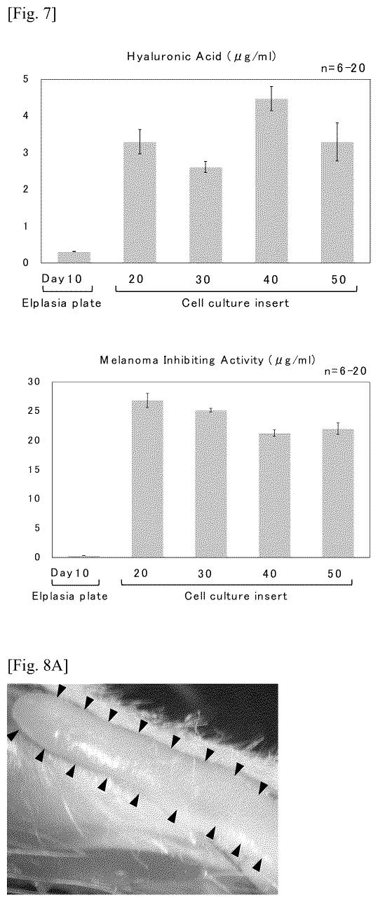

In ELISA, secretion levels of human hyaluronic acid and human melanoma inhibitory activity were measured every 10 days. It was confirmed that their secretion levels increased in shaped cartilage (at day 20 of culture) and that they could be maintained thereafter up to the stage of shaped hypertrophic cartilage (at day 50 of culture) (n=6−20) (FIG. 7).

Histological Analysis of Shaped Cartilage after Transplantation

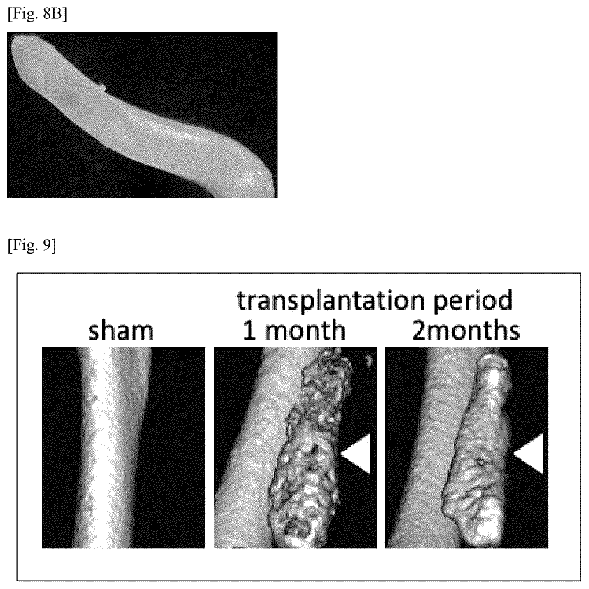

As a result of subcutaneous transplantation for 1 month, a shaped cartilage retaining its shape in macro images was obtained (FIGS. 8A, 8B).

### CT Image Analysis of Shaped Skeleton

As a result of subcutaneous transplantation of a shaped hypertrophic cartilage, an ossification image could be confirmed at the times of 1 month and 2 months after transplantation by CT imaging (FIG. 9).

### Histological Analysis of Shaped Skeleton

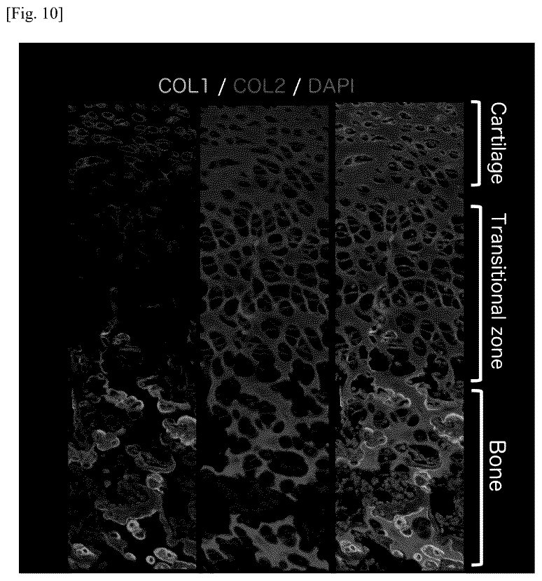

As a result of subcutaneous transplantation of a shaped hypertrophic cartilage, a bone-cartilage transitional zone and a COLI-positive trabecular bone could be confirmed by immunohistological staining at the time of 3 months after transplantation (FIG. 10).

Shaped Cartilage Using Chondrocyte Progenitor Cells Derived from Human Auricular Perichondrium

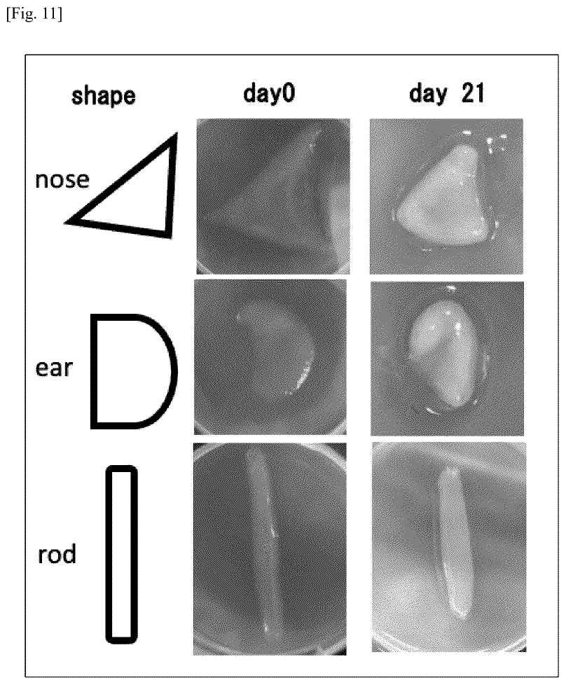

It was confirmed that a complex shape (mimicking nose, ear or rod in this Example) could be prepared even when chondrocyte progenitor spheroids derived from human auricular perichondrium were used (FIG. 11).

All publications, patents and patent applications cited herein are incorporated herein by reference in their entirety.

### INDUSTRIAL APPLICABILITY

The present invention is applicable to treatment of dyschondroplasia or the like in the craniofacial area, treatment of osteoarthritis or the like, and other aspects of regenerative medicine where shapes are considered important. The present invention is also applicable to orthopedic treatment. This version replaces all prior versions.

1. A method for creating an artificial cartilage tissue, comprising making chondrocyte progenitor cell-containing spheroids into a desired shape while seeding the spheroids on a support;
culturing the spheroids while feeding a culture medium from both the obverse and reverse sides of the spheroid-seeded surface to thereby perform fusion among the spheroids; and maturing the resultant fused spheroids into a cartilage tissue in vitro.

2. The method of claim 1, wherein the chondrocyte progenitor cell is a cell induced for differentiation from embryonic stem cells and/or induced pluripotent stem cells.

3. The method of claim 1, wherein the chondrocyte progenitor cell is a cell induced for differentiation from perichondrocytes collected from the perichondrium.

4. The method of claim 1, wherein the chondrocyte progenitor cell-containing spheroid is 20-1000 μm in diameter.

5. The method of claim 1, wherein one spheroid contains 100-7500 chondrocyte progenitor cells.

6. The method of claim 1, wherein the chondrocyte progenitor cell-containing spheroid has been prepared by culturing chondrocyte progenitor cells in a culture vessel having a non-cell-adhesive surface.

7. The method of any claim 1, wherein the chondrocyte progenitor cell-containing spheroid has been prepared by culturing chondrocyte progenitor cells in a culture medium containing TGF-B, bFGF and Wnt/B-catenin inhibitor.

8. The method of claim 1, wherein the fused spheroid is matured into a cartilage tissue by culturing in a culture medium containing BMP.

9. The method of Sclaim 1, wherein the time period of culture of the fused spheroid for maturing into a cartilage tissue is 14-42 days.

10. The method of claim 1, wherein the time period of culture of the fused spheroid for maturing into a cartilage tissue is 42-84 days.

11. The method of claim 1, further comprising transplanting the cartilage tissue matured in vitro into a non-human animal and maturing the cartilage tissue furthermore.

12. An artificial cartilage tissue prepared by the method of claim 1, which is 6 mm or more in diameter and 0.5 mm or more in thickness.

13. An artificial cartilage tissue prepared by the method of claim 10, wherein a part or the whole of the cartilage tissue is differentiated into bone tissue after transplantation into a living body.

14. A composition comprising an artificial cartilage tissue prepared by the method of claim 1, which is used for transplantation into a living body to compensate for a deficiency of cartilage tissue and/or bone tissue therein.

15. The composition of claim 14, which is used for transplantation into a living body to compensate for a deficiency of cartilage tissue therein.

16. The composition of claim 14, which is used for transplantation into a living body to compensate for a deficiency of bone tissue therein.

17. A method for preparing an artificial bone tissue, comprising transplanting an artificial cartilage tissue prepared by the method of claim 10 into a non-human animal and maturing the cartilage tissue into a bone tissue.

18. An artificial bone tissue prepared by the method of claim 17, which is 6 mm or more in diameter and 0.5 mm or more in thickness.

19. A composition comprising an artificial bone tissue prepared by the method of claim 17, which is used for transplantation into a living body to compensate for a deficiency of bone tissue therein.
